(19) United States
(12) Patent Application Publication
(10) Pub. No.: US 2013/0192375 A1
(43) Pub. Date: Aug. 1, 2013

(54) VOID DETECTION SYSTEM

(71) Applicant: GEORGIA INSTITUTE OF TECHNOLOGY, Atlanta, GA (US)

(72) Inventors: Kenneth Francis Barry, Concord, NC (US); Maria Silvia Guimaraes Biagini, Charlotte, NC (US); Juan Carlos Santamarina, Atlanta, GA (US); James Joseph Wall, Charlotte, NC (US); Yann Marcel Rene Le Pape, Huntersville, NC (US); John T. Lindberg, Huntersville, NC (US); Minsu Cha, Altanta, GA (US); Sheng Dai, Atlanta, GA (US); Seunghee Kim, Atlanta, GA (US)

(73) Assignee: GEORGIA INSTITUTE OF TECHNOLOGY, Atlanta, GA (US)

(21) Appl. No.: 13/755,724

(22) Filed: Jan. 31, 2013

Related U.S. Application Data
(60) Provisional application No. 61/592,877, filed on Jan. 31, 2012.

(51) Int. Cl. G01N 33/38

(52) U.S. Cl. CPC G01N 33/383
USPC 73/661; 378/54; 324/649

(57) ABSTRACT

A void detection system for detecting voids in fresh concrete is disclosed. The void detection system includes at least one probe disposed on a vibrator and adapted to detect voids in the fresh concrete, and an indicator in communication with the at least one probe and adapted to receive real-time data from the at least one probe. Upon receiving the real-time data, the indicator relays information to a user indicative of the fresh concrete's consolidation.

### BACKGROUND OF THE INVENTION

The present invention relates generally to a void detection system and, more particularly, to a void detection system for detecting voids in fresh concrete.

The degradation of concrete structures in nuclear power plants has been surveyed immediately following plant construction. The main types of degradation related to concrete are the existence of honeycombs and voids due to lack of adequate concrete placement and vibration.

Correct placement of concrete requires consolidation generally achieved by internal vibrators for normal weight concrete. Incorrect vibration can leave entrapped air in unconsolidated concrete. Other factors such as stiff or unworkable concrete, segregation, congested rebar, insufficient consolidation of concrete and inconsistent aggregate sizing can cause the formation of honeycombs and voids during concrete placement regardless of the quality of the vibration performed.

These types of problems are related to poor design and construction, which are the leading causes of age-related degradation in concrete structures, and have been described in NUREG 4652 (existing fleet). More recently, the NRC issued an Information Notice (2008-17) summarizing latest experiences and problems with concrete placement in other countries. These problems cause delays in construction due to the need to replace or repair concrete and influence the durability connected with a significant reduction of the service life. In parallel, maintenance and repair costs increase. The investigation of the early age concrete quality and detection of possible voids as honeycombs for insufficient compaction gains more importance the more complicated the construction processes are. Table 1 lists some of the facilities that have had honeycomb and/or void problems, and FIGS. 1-4 show defects caused by honeycomb and/or voids at existing facilities.

TABLE 1

| Plant | Year | Structure | Problem |
|---|---|---|---|
| San Onofre 1 | 1976 | diesel generator building center wall | voids 0.09 m2 7-10 cm penetration |
| Turkey Point 3 | 1968 | containment wall & reactor pit | voids |
| | 1982 | containment wall - equipment hatch | small void |
| Salem 2 | 1974 | containment - equipment hatch | incomplete pour & voids bc wrong concrete mix |
| Calvert Cliffs | 1971 | concrete near tendon bearing plates | voids caused plates to depress into concrete |
| ½ | | | |
| Three Mile | 1975 | wall of fuel handling building | void 7 cm × 1.8 m × 0.9-1.5 m bc improper |
| Island 2 | | | placement |
| Three Mile | 1976 | wall of fuel transfer canal | void 0.9-1.2 m × 0.4 m × 1.8-2.4 m |
| Island 2 | | | |
| Shoreham | 1974 | reactor support pedestal | voids and honeycombs in 1st lift |
| Brunswick ½ | 1974 | suppression chamber behind liner | voids |
| Beaver Valley 1 | 1982 | containment wall near equipment hatch | void 0.9 m × 0.9 m |
| Fermi 2 | 1984 | wall near door of Aux Building | voids |
| Waterford 3 | 1976 | foundation mat | cracks, honeycombs, cold joints improper |
| | | | placement not achieving stepped bedding planes |
| | 1976 | interior wall Aux bldg | voids, honeycombs, cold joints |

The detection of these honeycombs and voids in concrete structures has been attempted using non-destructive examination (NDE) technologies with limited success. The addition of Steel-Concrete (SC) construction techniques in new nuclear plants further complicates the ability of these technologies to reliably detect voids in even simple structural geometries.

Currently, the acceptance criterions for new construction are mainly based on visual inspection performed by an inspector on site. When hidden in the concrete mass voids are hardly detectable and when detected later as the structure ages they become the responsibility of the owner.

### BRIEF SUMMARY OF THE INVENTION

These and other shortcomings of the prior art are addressed by the present invention, which provides a void detection system capable of detecting voids in fresh concrete.

According to one aspect of the invention, a void detection system effective in detecting voids in fresh concrete includes at least one probe disposed on a vibrator and adapted to detect voids in the fresh concrete, and an indicator in communication with the at least one probe and adapted to receive real-time data from the at least one probe. Upon receiving the real-time data, the indicator relays information to a user indicative of the fresh concrete's consolidation.

According to another aspect of the invention, a void detection system effective in detecting voids in fresh concrete includes a vibrator adapted to vibrate fresh concrete and improve concrete consolidation, a plurality of probes disposed along a length of a main shaft of the vibrator, and an indicator in communication with the plurality of probes and adapted to receive real-time data from the plurality of probes. The plurality of probes is adapted to detect voids within the fresh concrete. Upon receiving the real-time data, the indicator provides a user with a Go/No-Go indication indicative of the consolidation of the fresh concrete.

According to another aspect of the invention, a method of preventing voids and/or honeycombing in fresh concrete includes the steps of providing a void detection system having at least one void detection probe disposed on a concrete vibrator, and an indicator in communication with the at least one void detection probe for providing a user with a Go/No-Go indication. The method further including the steps of positioning the at least one void detection probe in the fresh concrete at a first location, using the at least one void detection probe to test a consolidation of the fresh concrete and determine if voids are present, and using the indicator to receive data from the at least one void detection probe and provide a user with an indication indicative of the consolidation of the fresh concrete. If the indicator provides a Go indication to the user, the user moves the void detection system to a second location in the fresh concrete and tests the consolidation of the fresh concrete at the second location. If the indicator provides a No-Go indication to the user, the user uses the vibrator to vibrate the first location until a Go indication is received.

### BRIEF DESCRIPTION OF THE DRAWINGS

The subject matter that is regarded as the invention may be best understood by reference to the following description taken in conjunction with the accompanying drawing figures in which:

FIGS. 1-4 show defects caused by honeycomb and/or voids at existing facilities;

FIG. 5 shows a void detection system according to an embodiment of the invention;

FIG. 6 is a block diagram showing a method of detecting voids using the void detection system of FIG. 5.

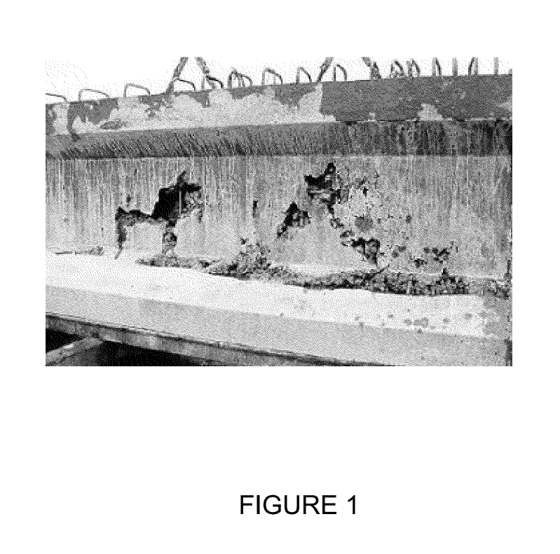

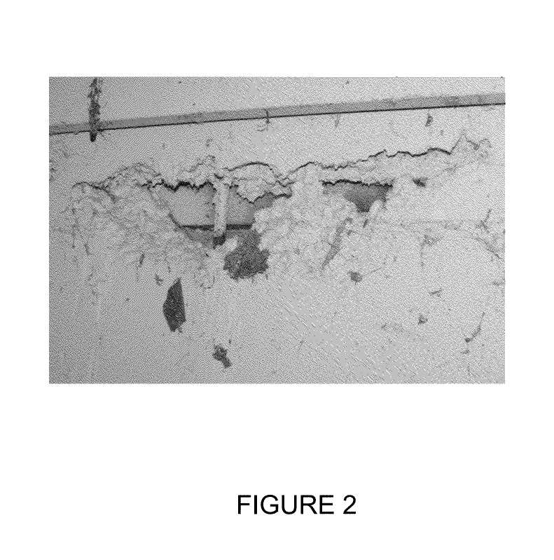

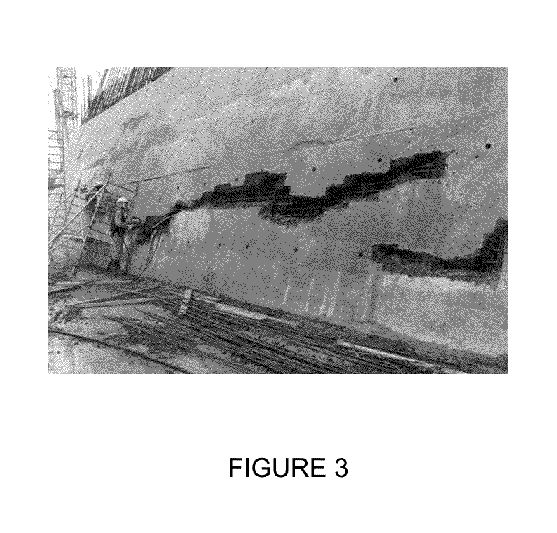

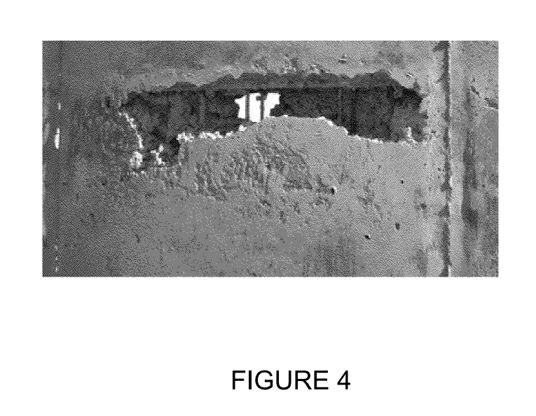

### DETAILED DESCRIPTION OF THE INVENTION

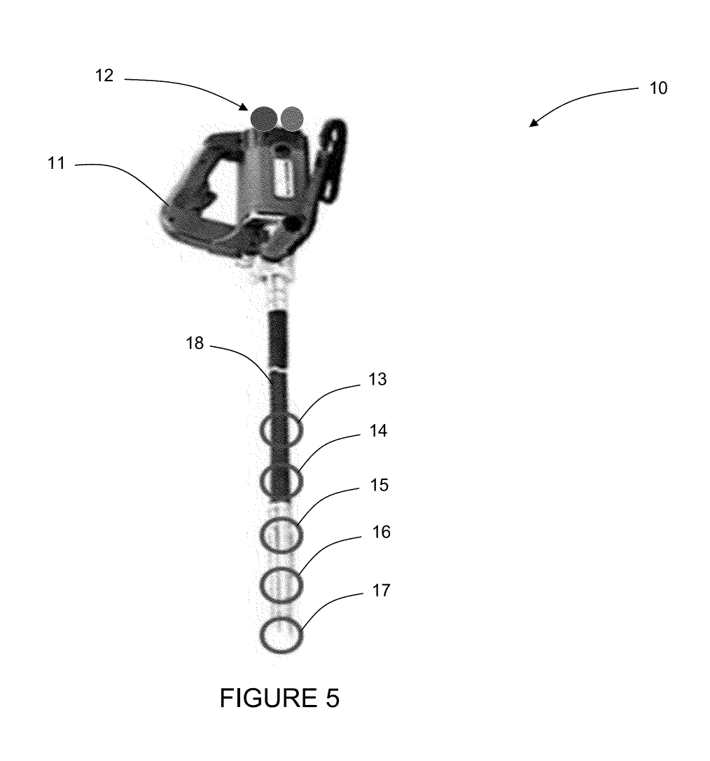

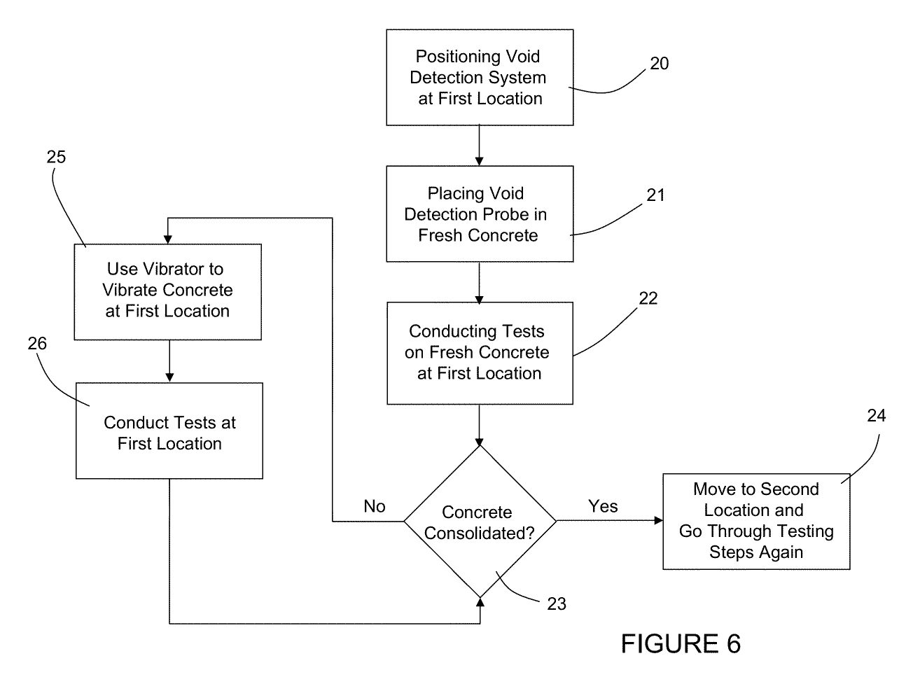

Referring to the drawings, an exemplary void detection system according to an embodiment of the invention is illustrated in the FIG. 5 at reference numeral 10. As shown, the void detection system 10 includes a concrete vibrator 11, a Go/No-Go indicator 12, and a plurality of probes 13-17 mounted on the vibrator 11 to detect voids within fresh concrete during placement and vibration. It should be appreciated that the detection system 10 may be a complete system including the concrete vibrator 11 or may be a commercially available vibrator 11 outfitted with the indicator 12 and probes 13-17.

The Go/No-Go indicator 12 is in communication (wired/wireless) with the probes 13-17 and may include a digital display or readout station for relaying information to a user, may be a plurality of lights (such as a red light for No-Go and a green light for Go), or may be audible signals that a user hears to provide a Go/No-Go signal. The indication will be based upon empirically determined thresholds to determine the Go/No-Go determination on the presence of voids.

The probes 13-17 are positioned along a main shaft 18 of the vibrator 11. The probes 13-17 present a combination of probes, including Gamma Density, Electrical Conductivity, P-Wave Velocity, and Time-Domain Reflectometry, to give real-time detection of concrete voids and honeycombs during concrete placement. The probes 13-17 provide information to the indicator 12 via signals for final display, thereby giving the user a Go/No-Go indication.

Time-Domain Reflectometry (TDR) Permittivity can measure a discontinuity in homogenous substances by observing reflected waveforms. At high frequency (upper MHz and GHz), the real permittivity decreases with increasing ionic concentration due to reduced mobility of the molecules involved in hydrating ions. Permittivity in fresh concrete is determined by volumetric water content and is affected by ionic concentration. There is a clear distinction between concrete and honeycomb.

P-Wave Velocity—Ultrasonic longitudinal waves propagate in gases, liquids or solids. P-Wave Velocity measurements are very different between fresh concrete and air. Therefore, they have a potential to have large signal to noise ratio.

While Electrical Conductivity of curing cement changes over time and is directly dependant on temperature; the presence of a void is distinctly discernable with an electrical probe. While this approach requires direct contact with the void, this technology can also be expanded to a resistivity tomography covering the area between two probes.

Gamma Density methods are based on selective absorption. Global absorption depends on the elemental absorption and mass fraction with amplitude decaying exponentially with distance ¹³⁷Cs is preferred for concrete-related applications.

In use, the detection system 10 is positioned in the fresh concrete at a first location (Block 20), placing the probes 13-17 into the fresh concrete (Block 21) such that the probes 13-17 are capable of running tests on the concrete, Block 22. If the tests show that voids are not present (Block 23), then the indicator 12 provides a Go signal and the user moves to a second location in the fresh concrete (Block 24) and begins the process anew. If the tests show that voids are present (Block 23), then the indicator 12 provides a No-Go signal and the user uses the vibrator 11 to work the voids out of the fresh concrete at the first location, Block 25. Tests are run again (Block 26) and a new determination is made as to whether voids are present in the concrete, Block 23.

The foregoing has described a void detection system for fresh concrete. While specific embodiments of the present invention have been described, it will be apparent to those skilled in the art that various modifications thereto can be made without departing from the spirit and scope of the invention. Accordingly, the foregoing description of the preferred embodiment of the invention and the best mode for practicing the invention are provided for the purpose of illustration only and not for the purpose of limitation.

We claim:

1. A void detection system effective in detecting voids in fresh concrete, comprising:
(a) at least one probe disposed on a vibrator and adapted to detect voids in the fresh concrete;
(b) an indicator in communication with the at least one probe and adapted to receive real-time data from the at least one probe, wherein upon receiving the real-time data, the indicator relays information to a user indicative of the fresh concrete's consolidation.

2. The void detection system according to claim 1, wherein the at least one probe is a gamma density probe.

3. The void detection system according to claim 1, wherein the at least one probe is an electrical conductivity probe.

4. The void detection system according to claim 1, wherein the at least one probe is a P-wave velocity probe.

5. The void detection system according to claim 1, wherein the at least one probe is a time-domain reflectometry probe.

6. The void detection system according to claim 1, wherein the void detection system includes a plurality of probes disposed on the vibrator, including a gamma density probe, an electrical conductivity probe, a P-wave velocity probe, and a time-domain reflectometry probe.

7. The void detection system according to claim 1, wherein the indicator provides a Go/No-Go indication to the user.

8. A void detection system effective in detecting voids in fresh concrete, comprising:
(a) a vibrator adapted to vibrate fresh concrete and improve concrete consolidation;
(b) a plurality of probes disposed along a length of a main shaft of the vibrator, the plurality of probes being adapted to detect voids within the fresh concrete;
(c) an indicator in communication with the plurality of probes and adapted to receive real-time data from the plurality of probes, wherein upon receiving the real-time data, the indicator provides a user with a Go/No-Go indication indicative of the consolidation of the fresh concrete.

9. The void detection system according to claim 8, wherein the plurality of probes are selected from the group consisting of gamma density probes, electrical conductivity probes, P-wave velocity probes, and time-domain reflectometry probes.

10. The void detection system according to claim 8, wherein the indicator includes a digital display adapted to provide the user with the Go/No-Go indication.

11. The void detection system according to claim 8, wherein the indicator includes a plurality of lights adapted to provide the user with the Go/No-Go indication.

12. The void detection system according to claim 8, wherein the indicator uses audible signals to provide a user with the Go/No-Go indication.

13. A method of preventing voids and/or honeycombing in fresh concrete, comprising the steps of:
(a) providing a void detection system having:
(i) at least one void detection probe disposed on a concrete vibrator; and
(ii) an indicator in communication with the at least one void detection probe for providing a user with a Go/No-Go indication;
(b) positioning the at least one void detection probe in the fresh concrete at a first location;
(c) using the at least one void detection probe to test a consolidation of the fresh concrete and determine if voids are present;
(d) using the indicator to receive data from the at least one void detection probe and provide a user with an indication indicative of the consolidation of the fresh concrete, wherein if the indicator provides a Go indication to the user, the user moves the void detection system to a second location in the fresh concrete and tests the consolidation of the fresh concrete at the second location, and wherein if the indicator provides a No-Go indication to the user, the user uses the vibrator to vibrate the first location until a Go indication is received.
